(19) United States
(12) Patent Application Publication
(10) Pub. No.: US 2015/0335905 A1
(43) Pub. Date: Nov. 26, 2015

(54) DEVICE FOR ORGANIC TISSUTAL RE-CONDITIONING, IN PARTICULAR FOR MALE OR FEMALE GENITALS

(71) Applicant: Enrico BONIZZONI, Busto Arsizio (IT)

(72) Inventor: Enrico BONIZZONI, Busto Arsizio (IT)

(73) Assignee: C.I.R.C.E. S.R.L., Magnago (Milano) (IT)

(21) Appl. No.: 14/408,853

(22) PCT Filed: Jun. 19, 2012
(86) PCT No.: PCT/IT2012/000187

§ 371 Date: Jan. 21, 2015

(51) Int. Cl. A61N 5/06

(52) U.S. Cl. CPC A61N 5/0616; A61N 5/0613; A61N 2005/0645; A61N 2005/0652; A61N 2005/0663; A61N 2005/0666; A61N 2005/0629

(57) ABSTRACT

A device (1) for organic tissutal re-conditioning comprises a support layer (2) associable with organic tissues, in particular vascularised tissues and still more particularly human male and/or female genitals; a plurality of light radiation sources (3) disposed on the support layer (2) and adapted to face the organic tissues to enable them to be irradiated, each source (3) being designed to emit radiation in a respective major frequency and according to a respective predetermined time interval; a control box associated with the sources (3) for controlling switching on and/or off of each source (3) according to a predetermined sequence and stimulating the organic tissues.

The present invention relates to a device for the so-called “organic tissutal re-conditioning”, for example concerning the tissues constituting the male or female genitals. In particular, the device according to the present invention is adapted to be used in treatments for curing the erectile dysfunction in the male, and for the photo rejuvenation of the external genitals in the female. The device can be also used in processes for treatment of the alopecia areata. In the following of the present description reference will be mainly made to the treatment of the erectile dysfunction, without however losing its general character.

It is known that the erection phenomenon is due to relaxation of the smooth musculature of the corpus cavernosum of the penis followed by arterial vasodilatation. The parallel constriction of the venous vessels causes blood stagnation followed by erection. Relaxation of the smooth muscles of the corpus cavernosum is a phenomenon mediated by nitrogen oxide. It activates the guanylate cyclase enzyme that catalyses conversion of guanosine triphosphate (GTP) into cyclic guanosine monophosphate (cGMP), that stimulates muscle relaxation. The cGMP is degraded by a phosphodiesterase (of which at least six isoenzymes are known). In the corpus cavernosum the involved phosphodiesterase is the phosphodiesterase type 5 (5PDE).

Under some physiological or pathological conditions, it is possible to incur a reduced functionality (or a quite absent functionality) of the genitals that therefore may not respond in an appropriate manner to stimuli connected with sexual arousal.

According to that which is known in the state of the art, for solving the problems mentioned above, resort is made to medicaments or drugs based on active ingredients such as the sildenafil, vardenafil and tadalafil for example, that are commercially known as Viagra, Levitra and Cialis, respectively; in particular, the sildenafil acts by inhibiting the 5PDE, which causes an increase in the blood afflux, following the increase in the cGMP concentration, giving rise to in improvement in the erection.

The known art mentioned above however has some drawbacks.

In greater detail, it is to be pointed out that the intensive use of these drugs involves problems connected both with the side effects on the whole blood circulation (or also, in some cases, the cardiovascular system) and with the so-called addiction phenomena, as a result of which a progressively growing amount of drug is gradually necessary.

In addition, the consumption of these drugs can be inhibited if the patient is under particular conditions, such as on occurrence of allergic conditions or if administration of other drugs incompatible with those specific to the cases in question are taking place.

It is also to be pointed out that the effect given by the drugs is generally temporary and therefore is inappropriate to solve pathological situations in the long run, which situations can instead become chronic and on the other hand give rise to high costs for supplying the drugs themselves.

Accordingly, it is an aim of the present invention to provide a device for organic tissutal re-conditioning overcoming the drawbacks found in the known art.

In particular, the present invention aims at making available a device for organic tissutal re-conditioning enabling application of therapeutic effects substantially free from side effects and/or addiction phenomena (or, on the contrary, effects of excessive sensitivity or allergenicity), which instead can be correlated with use of medicaments traditionally employed for these dysfunctions.

In addition, the present invention aims at providing a device that can be used also for a long period of time without important increases in costs by the patient, and that, furthermore, can give a significant help in finding a long-term solution to the physiopathological problems causing the tissutal dysfunctions.

Moreover, the present invention aims at making a device that can be indifferently used on patients of both sexes and that is as universal as possible in terms of anatomy and general physical conditions of the patients.

The technical task mentioned and the aims specified are substantially achieved by a device for organic tissutal re-conditioning comprising the technical features set out in one or more of the appended claims.

Further features and advantages of the present invention will become more apparent from the description, given by way of non-limiting example, of a preferred but not exclusive embodiment of a device for organic tissutal re-conditioning, as shown in the accompanying drawings.

In particular,

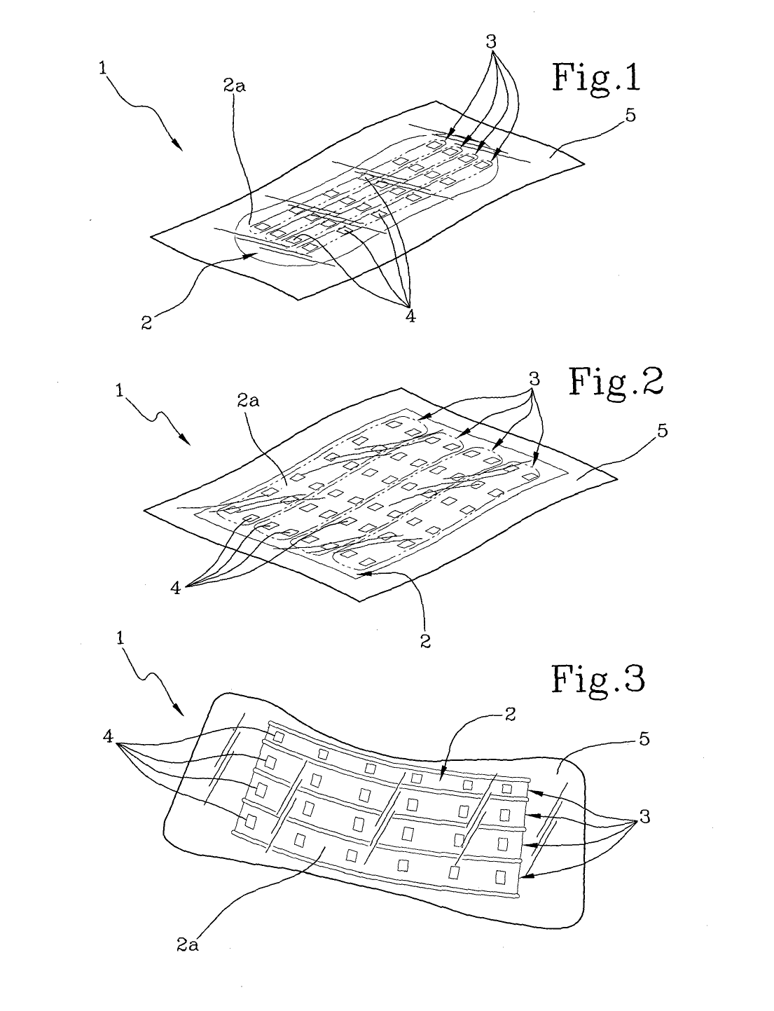

FIGS. 1, 2 and 3 show perspective views of a device for organic tissutal re-conditioning according to the present invention, in respective embodiments.

With reference to the drawings, a device for organic tissutal re-conditioning according to the present invention is denoted at 1.

Device 1 first of all comprises a support layer 2. This layer is associable with organic tissues, in particular vascularised tissues and still more particularly to human male and female genitals. In the context of the present description, by vascularised tissues it is intended tissues supplied with blood, also not in a continuative manner. By way of example, to the aims of the present invention tissues that are considered as vascularised are the scalp and the corpus cavernosum both of the penis and the clitoris.

In detail, the support layer 2 is flexible, and is preferably made of suitable material having an even thickness: for example, silicon rubber can be used or at all events a conveniently flexible and deformable polymer, that is also capable of ensuring the necessary hygienic conditions in contact with the organic tissues and to be maintained chemically/physically stable at the typical body temperature of a user.

The flexibility of the support layer 2 advantageously enables great versatility and adaptability of device 1. In particular, it makes device 1 able to adapt itself to the contact with any anatomic part.

The accompanying drawings show various possible forms of the support layer 2. In particular, FIG. 1 shows an elongated support layer 2. FIG. 2 shows a support layer in the form of a patch so that it is suitable for applications on areas to be treated that have different morphological features (for example adapted to be placed on a user's head for treating alopecia). FIG. 3 shows a support layer 2 designed to face the female genitalia.

Device 1 further comprises a plurality of light radiation sources. These sources 3 are disposed on the support layer 2, in such a manner as to face the organic tissues to be treated, so that they are irradiated.

In detail, each source 3 is provided for emitting radiation at a respective major frequency and a predetermined respective time interval. “Major frequency” (or only “frequency” in the following of the present disclosure) is understood as indicating the frequency of the irradiated radiation containing the major part of the emitted energy.

In particular, device 1 comprises at least one red source 3 intended for emitting radiation having a frequency included between 620 and 650 nm. Device 1 further comprises at least one green source intended for emitting radiation having a frequency included between 490 and 530 nm. Device 1 also comprises at least one yellow source 3 intended for emitting radiation having a frequency included between 540 and 560 nm. Device 1 comprises at least one blue source intended for emitting radiation having a frequency included between 450 and 470 n.

Finally, it should be noted that in alternative embodiments, device 1 may comprise one or more of the above listed source types. In addition device 1 may also comprise sources 3 belonging to types not listed above, capable of emitting electromagnetic radiation at a frequency that can be extended in the range from the infrared to the ultraviolet.

Following experimental considerations, it has been stated that a light flux of 1030 mlm, produced by the assembly of sources 3 of device 1 is advantageous for producing the above mentioned therapeutic effects. This light flux can at all events show variations of the cyclic or transient type during activation and/or deactivation of sources 3. Also taking into account these effects, the mean value of the light flux produced by sources 3 can be varied and adjusted to the specific application of device 1, as well as to the patient's physiology. It should be finally noted that in the preferred embodiment, sources 3 are activated one at a time in sequence, as better specified in the following of the present specification. In this case, each source 3 will be able to produce the whole light flux, i.e. the already mentioned value of 1030 mlm.

In detail, each light source 3 comprises a plurality of elements 4 capable of emitting radiation at the predetermined frequency of source 3. These elements 4 are, by way of example, light-emitting diodes or LEDs.

As highlighted in the accompanying figures, elements 4 composing sources 3 are disposed like a grid on support 2. In the embodiments shown elements 4 composing each source 3 are gathered, i.e. each source 3 is placed on a respective area of the support layer 2. In alternative embodiments of the invention, elements 4 composing different sources 3 are alternated with each other, i.e. each source 3 is distributed over the whole surface of the support layer 2.

In greater detail, in both embodiments, elements 4 are separated from each other by a distance included between 1 and 2 cm. Alternative embodiments not shown are also possible in which elements 4 are disposed on the support layer 2 following different modalities.

Device 1 further comprises one or more power supply circuits (not shown) for sources 3. These circuits are connected to one or more power supply sources.

Device 1 according to the present invention further comprises a control box (not shown) associated with sources 3. The function of this control box is to control switching on and/or off of each source 3 following a predetermined sequence and, in this manner, suitably stimulate the organic tissues.

In detail, the control box is configured to activate the red source 3 during an interval included between 270 and 330 sec, preferably equal to 300 sec. The control box is further configured to activate the green source 3 during an interval included between 210 and 270 sec, preferably equal to 240 sec. The control box is also configured to activate the yellow source during an interval included between 330 and 390 sec, preferably equal to 360 sec. Finally, the control box is configured to activate the blue source during an interval included between 150 and 210 sec, preferably equal to 180 sec.

In more detail, the control box activates each source 3 following pulses of a duration of 50 msec. Preferably, the control box waits for 70 msec between two consecutive pulses.

The activation sequence is the following: the control box first activates the red source 3. After activation (and preferably immediately after deactivation) of the red source 3, the control box activates the green source 3. The control box goes on in the same manner activating the yellow source 3 after the green source 3 and the blue source 3 after the yellow source 3.

In a preferred embodiment, device 1 comprises a reflecting layer 2a overlapping the support layer 2. This reflecting layer 2a, in use, faces the organic tissues, so that it concentrates the light radiation emitted from sources 3 onto the organic tissues.

Device 1 may further comprise a housing 5 fully wrapping the support layer 2 and sources 3. In particular, the function of housing 5 is to sealingly insulate sources 3. Advantageously, this solution enables device 1 to be washed, sanitised and/or disinfected.

In a first embodiment, housing 5 is made by casting of transparent silicon resin. By this casting operation at least the support layer 2 and sources 3 are fully incorporated. According to an alternative embodiment of the invention, housing 5 is made by wrapping the support layer 2 and sources 3 within heat-sealable plastic material films.

Should sterilisation of device 1 be necessary (in case of treatment of diabetic ulcer for example, or following tissutal implants for promoting attachment of same), device 1 comprises a sterile bag (not shown) made of transparent plastic material. This sterile bag directly wraps the support layer 2 and sources 3 or housing 5, if any. Advantageously, if sterility of device 1 should be impaired, for restoring it, it is only necessary to replace the sterile bag.

Advantageously, the above described device 1 can be sewn or directly fastened to a garment, preferably to underwear. It is therefore also part of the present invention an item of underwear comprising a portion of textile material shaped like a predetermined anatomic region of a user, to which device 1 is applied.

It is also part of the present invention a kit comprising the above described device. This kit further comprises an ointment for topical use the active ingredient of which is different depending on the effect to be achieved. By way of example, this ointment may comprise a vegetable extract of Serenoa Repens to treat alopecia.

Alternatively, the ointment may comprise L-arginine for the above-mentioned therapeutic treatments on both male and female genitals. In addition, a version of the ointment applicable to the female genitals may also comprise lactic acid.

Advantageously, use of the kit reaches a synergistic effect because the light emitted by device 1 promotes absorption of the active ingredient present in the ointment, by the organic tissues treated. This effect is added to the individual effects of the ointment and of device 1 that at all events are present.

The invention achieves different interesting advantages.

First of all it should be noted that the base construction architecture of the above described object (as hereinafter claimed) enables a curative function to be exerted on the tissues that is not necessarily based on an active ingredient of chemical nature; as a result, side effects can be avoided and it is possible to set up a long-term therapeutic system with results that can therefore overcome the only temporary effectiveness typical of treatments based on the currently most used drugs in this field.

It should be in addition highlighted the fact that application of the present device in close contact with the tissues to be treated enables the patient to remain under comfortable conditions for use and movement and also enables easy adaptability to any type of anatomy or general physical condition (to the benefit of the flexibility of application and therefore offering the possibility of producing a very limited number of variants of the device itself).

Finally, it will be recognised that the present invention combines together low manufacturing costs, great ease of use and/or replacement in case of breaking and long operating lifetime.

1. A device (1) for organic tissutal re-conditioning, characterised in that it comprises a support layer (2) associable with organic tissues, in particular vascularised tissues and still more particularly human male and/or female genitals; a plurality of light radiation sources (3) disposed in said support layer (2) and adapted to face said organic tissues to enable them to be irradiated, each source (3) being designed to emit radiation according to a respective major frequency and a respective predetermined time interval; a control box associated with said sources (3) for controlling switching on and/or off of each source (3) in a predetermined sequence and stimulate said organic tissues.

2. A device (1) as claimed in claim 1, wherein said plurality of sources (3) comprises at least one red source (3) designed to emit radiation of a frequency included between 620 and 650 nm and/or at least one green source (3) designed to emit radiation of a frequency included between 490 and 530 nm and/or at least one yellow source (3) designed to emit radiation of a frequency included between 540 and 560 nm and/or at least one blue source (3) designed to emit radiation of a frequency included between 450 and 470 nm.

3. A device (1) as claimed in claim 2, wherein said control box is configured to activate said red source (3) during an interval included between 270 and 330 sec, preferably equal to 300 sec, and/or said control box is configured to activate said green source (3) during an interval included between 210 and 270 sec, preferably equal to 240 sec, and/or said control box is configured to activate said yellow source (3) during an interval included between 330 and 390 sec, preferably equal to 360 sec, and/or said control box is configured to activate said blue source (3) during an interval included between 150 and 210 sec, preferably equal to 180 sec.

4. A device (1) as claimed in claim 3, wherein said control box is configured to activate each source (3) according to pulses having a duration of 50 msec, said control box being further configured to wait for 70 msec between two consecutive pulses.

5. A device (1) as claimed in claim 2, wherein said control box is configured to activate said green source (3) immediately after deactivation of said red source (3); said control box being configured to activate said yellow source (3) immediately after deactivation of said green source (3); said control box is configured to activate said blue source immediately after deactivation of said yellow source (3).

6. A device (1) as claimed in claim 1, wherein each source (3) is designed to emit a light flux of 1030 mlm.

7. A device (1) as claimed in claim 1, further comprising a reflecting layer (2a) overlapping said support layer (2) and facing said organic tissues to concentrate said light radiation emitted from said sources (3) on said organic tissues.

8. A device (1) as claimed in claim 1, wherein each source (3) is defined by a plurality of elements (4), preferably light emitting diodes or LEDs, separated from each other by a distance included between 1 and 2 cm.

9. Kit characterised in that it comprises a device (1) as claimed in claim 1 to enable organic tissues to be irradiated, in particular human male and/or female genitals; a ointment to be rubbed on said organic tissues.

10. Underwear comprising a portion of textile material shaped according to a predetermined anatomic region of a user, characterised in that it comprises at least one device (1) as claimed in claim 1 applied to said portion of textile material and adapted to face an organic tissue of said user.
